(19) United States
(12) Patent Application Publication
(10) Pub. No.: US 2024/0402071 A1
(43) Pub. Date: Dec. 5, 2024

(54) Two-Phase Adhesive Nonstandard COSA Devices

(71) Applicant: BVW Holding AG, Cham (CH)

(72) Inventors: Lukas Bluecher, Eurasberg (DE); Michael Milbocker, Holliston, MA (US)

(21) Appl. No.: 18/624,992

(22) Filed: Apr. 2, 2024

Related U.S. Application Data
(60) Provisional application No. 63/456,999, filed on Apr. 4, 2023.

(51) Int. Cl. G01N 19/02

(52) U.S. Cl. CPC G01N 19/02

(57) ABSTRACT

A device with a microstructure arrangement for gripping low coefficient of friction material which may include a set of microfeatures disposed on a substrate. The microfeatures may provide a physical property of a grip force exceeding a grip force theoretically achieved by friction theory alone. The microfeatures may provide a grip force in excess of 50.0 N. The microstructure arrangement may include a gripping surface with a contact area of 25% or less of the total macroscopic area of the device.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application claims benefit of the following patent application(s) which is/are hereby incorporated by reference: U.S. Provisional Patent Application No. 63/456,999 filed Apr. 4, 2023

### STATEMENT REGARDING FEDERALLY SPONSORED RESEARCH OR DEVELOPMENT

### Not Applicable

### REFERENCE TO SEQUENCE LISTING OR COMPUTER PROGRAM LISTING APPENDIX

Not Applicable

### BACKGROUND OF THE INVENTION

The present invention relates to microstructure patterns and, more particularly, to a gripping surface having a microstructure pattern adapted for improving grip on low coefficient of static adhesion (COSA) materials, which does not rely on changing the COSA of the bulk material.

Polymer materials with low COSA, such as PTFE, HDPE, nylon, PET, PLA, and POM are manufactured into devices to allow for such devices to slide or be translated easily across a surface, generally without a lubricant being present on the device or the surface, or both.

Examples known in the art having low COSA include a PTFE shaft seal that can slide against a polymeric housing; a nylon polymer covered vascular catheter sliding through a polypropylene introducer tube; or a PTFE coated guide wire sliding inside a polymer lined vascular catheter. Often, the material can have a coefficient of friction of 0.2 or less when the material is tested under standard conditions.

However, though the low COSA of a material may be an advantage under some conditions, it is likewise a disadvantage under others. For instance, low COSA materials can be difficult to handle and manipulate during manufacturing, during use of the material, or during disassembly. Previous attempts to provide sufficient adhesive force to overcome the issues with low COSA materials have included the use of additives to the material that results in “sticky” surfaces. Other attempts include the addition or use of adhesives to provide sufficient gripping force during handling or manipulation of the low COSA material. However, such previous attempts are unsatisfactory because the additives or adhesive result in undesirable materials as they can be too sticky, create unnecessarily rough surfaces, and can cause discomfort when handled or used.

Thus, there is a need to create a surface which can grip low COSA materials that allow for easier handling of the material but yet do not damage the material surface. Surface texture that is intended to be frictional typically damages the target surface by mechanically engaging with the surface. Therefore, increasing the grip force by increasing friction typically results in the surface of the material being damaged, which inhibits and/or limits the low COSA characteristics of the material.

Accordingly, it is an object of the present disclosure to provide surface having a microstructure pattern to create a gripping surface that may be capable of gripping low COSA materials with greater force than a non-patterned surface and further, without causing damage to the low COSA material.

### BRIEF DESCRIPTION OF THE SEVERAL VIEWS OF THE DRAWINGS

FIG. 1 is an illustration of an embodiment of an apparatus for determining the coefficient of static adhesion (COSA) of a material.

FIG. 2 is an illustration of an embodiment of a basic two-level microstructure and method of modification.

FIG. 3 is an illustration of an embodiment of a hierarchical and lateral arrangement of a microstructure surface.

FIG. 4. is an illustration of an embodiment of a microstructure surface depicting the contact angle and parameters affecting surface energy.

FIG. 5 is an illustration of a PET polymer used in the manufacture of microstructured devices.

FIG. 6 is an illustration of a poloxamer polymer used in the manufacture of microstructured devices.

FIG. 7 is an illustration of an embodiment of a microstructured device where some of the microstructure has a portion that is hierarchical and a portion that is lateral.

FIG. 8 is an illustration of an embodiment of a microstructured device where one level of microstructure mirrors the symmetry of another level of microstructure.

FIG. 9 is an illustration of an embodiment of a microstructured device wherein one microstructure comprises a second microstructure.

FIG. 10 is an illustration of an embodiment of a microstructured device employing a microstructure designed to provide a Wenzel-Cassie adhesion effect and another microstructure designed to provide a frictional effect.

FIG. 11 is an illustration of an embodiment of a microstructured device employing a microstructure designed to provide a suctional effect and another microstructure designed to provide a sealing effect.

### DETAILED DESCRIPTION OF THE INVENTION

With reference to the drawings, various embodiments will now be described in more detail. Unless defined otherwise, all technical and scientific terms used herein have definitions consistent with their respective disciplines. Methods, devices, and materials similar or equivalent to those described herein can be used in the practice or testing of the presently disclosed subject matter, representative methods, devices, and materials are described herein.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of the items be present in the grouping. Furthermore, “and” is to be regarded as “and/or” except in the claims, and unless expressly stated otherwise.

Similarly, a group of items linked with the conjunction “or” should not be read as requiring mutual exclusivity among the group, but rather should also be read as “and/or” unless expressly stated otherwise.

Furthermore, although items, elements or components of the disclosure may be described or claimed in the instant examples, the plural or combinations of such are contemplated to be within the scope thereof unless limitations to the singular is explicitly stated. The presence of broadening words and phrases such as “one or more”, “at least”, “but not limited to” or other like phrases in some instances shall not be read to mean that the narrower case is intended or required in instances where such broadening phrases may be absent.

The gripping force generated by the present devices may, in some embodiments, exceed the grip forces due to friction alone, especially when the interface between two surfaces contains a liquid. This higher wet gripping force seen in some embodiments may be achieved by a suctional aspect to the interface between the device and the target surface. For example, some embodiments of the microstructured surfaces of the present disclosure may include a Wenzel-Cassie interface, as well as appreciable capillary forces due to the hierarchical arrangement of the microstructure.

Other aspects contributing to the improved gripping force of embodiments described in the present disclosure may include high contact perimeter, and a combination of hydrophobic and hydrophilic regions of microstructure that may promote adhesion.

In some embodiments disclosed herein, a microstructured device may be capable of generating high static adhesion, rather than high kinetic adhesion. Kinetic adhesion is generally mediated by mechanical coupling between the microstructured surface and a target surface. Additionally, in some embodiments, the microstructured surface may be capable of generating high COSA.

In some embodiments, the microstructured device may comprise a COSA that is greater when there is liquid present between the device and target surface compared to when there is no liquid present between the device and target surface.

In certain embodiments, a microstructured surface may comprise a surface pattern which may be applied to a material intended to grip a target surface. In certain embodiments of the microstructured surface, the microstructure may include microfeatures which may include protuberances and/or recessions which may be stacked hierarchically. In some embodiments, the microfeatures may be disposed longitudinally in relation to one another. The microfeatures may be of different sizes. In some embodiments, the microfeatures may be arranged in a hierarchical pattern wherein a first set of microfeatures are larger than a second set of microfeatures and wherein the larger set of microfeatures support or augment the effects of the second set of microfeatures.

The hierarchical arrangement of the microstructure pattern may be scale or size dependent, surface energy dependent, or functionally dependent. In such an arrangement, a first microfeature may support the effects of a second microfeature. Additionally, in some embodiments, a hierarchical pattern will be understood to include one structure that resides on the surfaces of a second structure, but the present disclosure of embodiments is not to be limited to this interpretation alone.

In certain embodiments of the present disclosure, the pattern of microstructures arranged as disclosed herein, hierarchically or laterally, may induce a first shear force. It will be understood that the first shear force may comprise much more than a frictional force as defined in the standard physics literature. In particular, the microstructure layers or scales of size may be arranged to generate domains of -philic and -phobic interface regions, that may be described as being hydrophilic or hydrophobic, but may also be lipo-, oleo-, and the like regarding philicity and phobicity. In some embodiments, these philic-phobic domains may generate shear forces that far exceed classic physical frictional forces of the same or similar material.

In some embodiments, the microstructure pattern may be arranged such that two adjacent microfeatures have a pitch defined as the percent of the diameter, width, and/or thickness of one, or the other, or both of the microfeatures. For example, in one embodiment, the microfeatures may include pillars with a diameter of 10 microns which may be spaced 1 micron apart, thus having a pitch of 10%.

In some embodiments, there may be an adhesive force associated with surfaces that have a microstructure pattern, where the adhesive force is greater than the expected frictional force. This adhesive force may be achieved even when the frictional component is marginally enhanced. It will be recognized that most frictional microstructure mediated forces have a coefficient of friction between 0.7 and 1.4.

The coefficient of friction as used herein may be understood to include the normal applied force divided by the shear force required to move a surface with respect to a contact surface. Typically, the shear force F is related to the normal force N by a coefficient of friction (COF) μ. Such a relationship can be defined as F=μN where the contact area between the object and the target surface is equal. Thus, μ may be understood as a linear function of contact area.

In certain embodiments disclosed herein, where the contact area is not known, the coefficient of friction may be measured as if the contact area is one-to-one. For a microstructured surface, that surface contact area may be less than 1, and indeed the surface contact area is inversely proportional to the coefficient of friction. Thus, a surface contact area that is 1 to 1 may have a coefficient of friction u=1. However, the same surface with a microstructure pattern where the contact area is decreased, will typically have a coefficient of friction proportional to the reduced area. For example, for a material possessing a coefficient of friction equal to 1, if the contact area is reduced by microstructure patterning by a factor of 2 then the coefficient of friction may be equal to or less than 0.5.

Alternatively, the same microstructure pattern may also increase the coefficient of friction due to edge effects associated with the microstructures, e.g., the coefficient of friction for the surface with a microstructure may be greater than 1. Consequently, a micropattern may possess a high coefficient of friction or low coefficient of friction relative to a surface of the same material with no microstructure.

It is important to recognize that the coefficient of friction of any material may be modified in the range of 0.5 to 1.5 generally, by a simple increase of the perimeter length (not an area) or a decrease in surface area contact.

Contact surface area as used herein may be understood to include the area of contact between a microstructured surface and a target surface when both surfaces are dry and oriented perpendicular to the direction of gravity.

The contact surface area of a microstructured surface and target surface when the target surface is wet may be understood as the contact surface area when there is no liquid between the microstructured surface and target surface.

It should be understood that edge effects have long been known in the prior art, and devices incorporating increased perimeter length to achieve a higher coefficient of friction are not novel. Various embodiments disclosed herein may not rely solely on increased perimeter length for their adhesive properties. In the present disclosure, edge effects and surface area contact comprise a perturbation on a more fundamental and stronger effect.

It is important to recognize that an outer surface of a gripping pad comprising a microstructured pattern that includes, for example, micropillars, or alternatively, microstructure recesses, may exhibit a high grip force and high coefficient of friction relative to the same material which is without a microstructure pattern. The present disclosure relies on the juxtaposition of microfeatures with different surface energies to achieve a higher adhesion to a target surface. This increase in adhesion may occur when one or the other or both surfaces are dry or wet.

In some embodiments, a microstructured surface of a polymer (for example, PTFE) may be disposed on a mold of metal, e.g. silicon, wherein the polymer may have a coefficient of friction relative to the metal of 0.8 or higher. The same polymer surface without the microstructured pattern disposed on a mold of metal, e.g. silicon, may have a coefficient of friction of about 0.2. Thus, the microstructured patterned polymer may have a higher COF compared to the non-patterned polymer. It will be understood that the coefficient of friction is usually defined between two substances where the contact area is infinite, and the effect of the perimeter is minimal.

For microstructured surfaces, the contact area may be minimized relative to the length of the perimeter for each microstructure surface of contact. It should be understood that the microstructure perimeter length may be the perimeter of a microstructure cross section. It should also be understood that the microstructure perimeter length of a microstructure set may be the product of the microstructure perimeter length of one microstructure and the number of microstructures in the microstructure set. It should also be understood that a microstructured device perimeter length may be the sum of the perimeter lengths of the microstructure sets.

In certain embodiments, the ratio between perimeter and contact area may enable a change in coefficient of friction where no such change actually occurs, if the effect of the increase in the contact perimeter is factored in.

When the perimeter length is constant when comparing materials, the coefficient of friction has a standard meaning. When the perimeter is increased even microscopically, then one may no longer rely on the simple equation given above, F=μN. In particular, the coefficient of friction may vary strongly with the perimeter length relative to the contact area. It will be appreciated that this relationship is known in the prior art. However, improvement in resistance to translation due to a perimeter effect is not the subject of the present disclosure and its embodiments.

Prior art devices which include a microstructure pattern formed on a pad of high modulus polymer, such as polyester terephthalate (Mylar) or modified polyester terephthalate (Hytrel or Tritan) which may result in an increase in COF of polyester terephthalate to PTFE from about 0.04 to over 1.00. This effect may be fully explained by the increase in the perimeter length due to the microstructure, as illustrated in Table 1. Table 1 shows a linear relation between the coefficient of friction and the perimeter length of the individual microstructures.

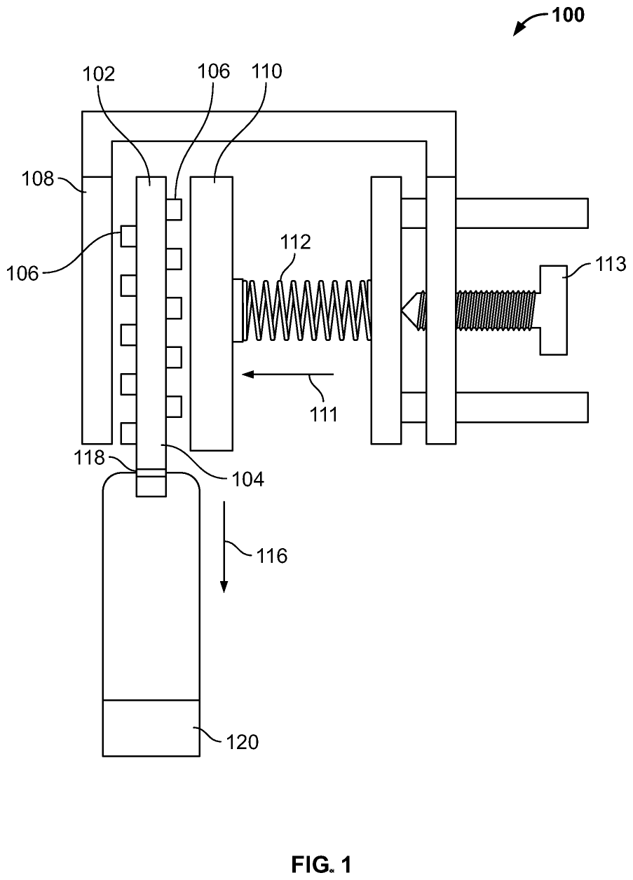

To measure the COSA (u), a testing apparatus was used as illustrated in FIG. 1. The COSA method and device 100 comprise the following: a sample 102 comprises a surface 104 which includes microstructure features 106 on both sides of the surface 104; said surface and microstructure features may be the same size as the first and second gripping surfaces 108, 110; the first gripping surface 108 is fixed in position whereas the second gripping surface 110 applies a normal force (N) 111, which is adjusted via an adjuster 113 and tension spring 112; wherein the applied force can be measured with a strain gauge, which is subsequently removed before applying a weight 120. The applied normal force (N) measurement is procured by placing the sample 102 comprising surface 104 and microstructure features 106 between gripping surfaces 108, 110 and pulling in direction 116 until the pulling force causes the sample 102 to become displaced between the gripping surfaces 108, 110. The sample surface 104 also includes a hole 118 to which can be attached the weight 120. The COSA (μ) is measured by connecting the weight (50 Newton force total) 120 to hole 118 and reducing the applied force supplied by tension spring 112 until the sample 102 falls from the gripping surfaces 108, 110 and subsequently measuring the normal force 111 with strain gauge 114. The COSA (μ) is calculated using the following equation:

$${F}_{weight} = μ \times N \to μ = \frac{50 Newtons}{2 \times ( spring force )}$$

It is important to note the factor of 2 in the denominator. This factor is due to the two-sided sample comprising surface 104 and microstructure features 106.

A test setup as depicted in FIG. 1, but which uses a sample comprising a substrate with only one pattern on one side, is in actuality measuring a COSA which is the mean of the COSA of the two surfaces, one with microstructured surface and the other a smooth surface. Such an experimental setup does not measure a COSA or COF of the dual-sided microstructured surfaces 106. It should also be noted that an experimental setup as in FIG. 1 is insufficient for measuring a kinetic coefficient of friction.

Referring to the literature, AIP Handbook, Coefficients of Friction, Dudley Fuller, the kinetic coefficient of friction is the magnitude of maximal frictional force divided by the magnitude of the normal thrust once any motion is established. The problem with the kinetic definition is that the kinetic coefficient of friction depends on atmospheric humidity, velocity of sliding, temperature, vibration, and any surface contaminants, whereas these factors are insignificant when determining the static coefficient of friction.

For example, the AIP Handbook gives the following (Table 1) kinetic coefficients of friction.

TABLE 1
Kinetic Coefficients of Friction, Steel on Steel, Unlubricated

| Velocity (in/sec) | 0.0001 | 0.001 | 0.01 | 0.1 | 1 | 10 | 100 |
|---|---|---|---|---|---|---|---|
| Kinetic COF | 0.53 | 0.48 | 0.39 | 0.31 | 0.23 | 0.19 | 0.18 |

The data above indicates that the kinetic coefficient of friction varies differently as a function of sliding velocity for different materials. There is no consistent kinetic COF measurement that can be used to compare different materials with different microstructured surfaces, unless the ambient conditions are precisely defined and maintained.

The static coefficient of friction is a single-valued quantity which can take place in a wide variety of ambient conditions, provided the sample being measured is not in motion, i.e., the measurement is made just prior to slippage.

Table 2 is an example of an effect which is generally applicable to simple surface microstructured patterns. It should be recognized that a hierarchical pattern, for example, where one set of microfeatures are disposed on top of a second set of microfeatures, generally applies to the top most microstructure, only. Consequently, any effect can classically depend on deformation of the microstructure caused by the supplied normal force, and thus is not expected to be linear as the equation given above implies. Nevertheless, it is maintained that all of these effects are well-known regarding an accurate computation of the edge effects quantified by the perimeter length.

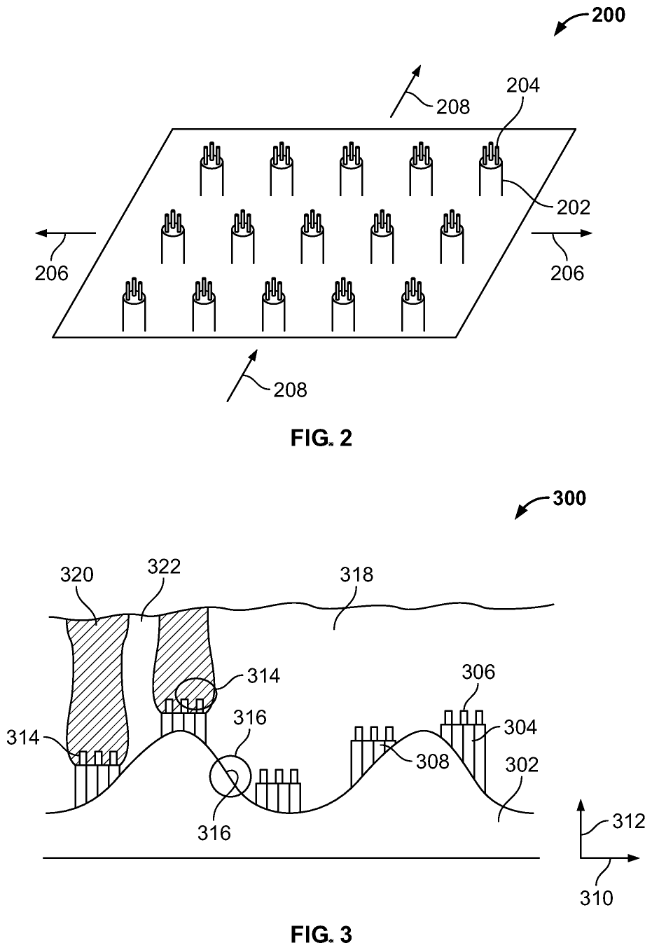

Table 2 is the result of COSA (μ) measurement of an embodiment having a microstructure surface as depicted in FIG. 2. The microstructure surface 200 may include first microfeatures 202 and second microfeatures 204 wherein the second microfeatures are disposed on the first microfeatures. In some embodiments, the first microfeatures 202 may have a diameter of about 200 microns. The second microfeatures 204 may have a diameter of about 20 microns. In order to vary the perimeter length, strips of a substrate were embossed on top of the microstructure. In some embodiments, microstructure surfaces were molded from polylactic acid.

It should be appreciated that when referring to “100% microstructure” it would include an example where 200 micron pillars are spaced 100 microns over the entire substrate surface. Whereas “90% microstructure” means 10% of microstructure is embossed with a flat surface. Surfaces with microstructure covering the substrate from 100%-10% were tested. The minimum contact area between microstructured surface and a target flat surface is provided by the 100% microstructured surface and the contact area increases as microstructure area decreases.

TABLE 2
COSA, Dry (N = 10)

| Microstructure(area of contact) | COSA (shear force/normal force) |
|---|---|
| PLA sheet no microstructure | 0.23 +/− 0.09 |
| 100% area of contact | |
| PLA sheet 100% | 1.08 +/− 0.19 |
| microstructure <25% | |
| area of contact | |
| PLA 90% | 0.87 +/− 0.11 |
| PLA 80% | 0.65 +/− 0.25 |
| PLA 70% | 0.56 +/− 0.22 |
| PLA 60% | 0.47 +/− 0.19 |
| PLA 50% | 0.41 +/− 0.16 |
| PLA 40% | 0.39 +/− 0.11 |
| PLA 30% | 0.35 +− 0.19 |
| PLA 20% | 0.29 +/− 0.09 |
| PLA 10% | 0.27 +/− 0.08 |

The static coefficient of friction of a microstructured surface may depend both on the surface area of contact (as illustrated in Table 2) and the length of the perimeter. The area of contact is well described, and static coefficient of friction may be linearly proportional to the surface area of contact. In Table 2, the surface area of contact and the perimeter of the microstructure may be reduced by the same amount.

Referring to FIG. 2, a microstructure surface may be stretched in a first direction 206 and compressed in a second direction 208 so that the density of first microfeatures 202 may be varied and the surface area may be independently increased. The surface area of microstructure surface 200 may be increased by compressing second microfeatures 204 to alter the arrangement of the microstructure features on the surface. The surface area of contact may be the sum of the first microfeatures 202 surface areas. The perimeter length of the first microfeatures 202 may be the circumference of the newly arranged surface. The perimeter length of contact is the sum of the perimeter lengths of the first microfeatures 202.

When the density of pillars is reduced, the perimeter may be increased to maintain the contact surface area. In Table 3, the surface area is constant and the perimeter is varied, where the COSA measurements are normalized to the pattern of Table 2 with a surface area 1 and perimeter length of 1.

TABLE 3
COSA, Dry

| Contact Area | Perimeter area | COSA |
|---|---|---|
| 1 | 1 | 1.00 +/− 0.15 |
| 1 | 0.8 | 0.81 +/− 0.25 |
| 1 | 0.5 | 0.69 +/− 0.18 |
| 1 | 0.3 | 0.41 +/− 0.22 |

The above table illustrates that about 75% of the reduction in static coefficient of friction may be due to the perimeter length and not the surface of contact. Hence, using the standard equation for the static coefficient of friction or COSA (for microstructured surfaces), the surface area of contact may be relatively unimportant relative to the length of the perimeter of the microstructure. Thus, two microstructures with the same area of contact, but one with large pillars and the other with small pillars, the small pillar microstructure may have the higher COSA.

Consequently, a microstructured pattern described in FIG. 1 may be formed of a pad of steel or high modulus polymer that grips any lower modulus polymer material with a COF greater than 0.8. In the present coefficient of friction measurement protocol, it is not unexpected that high modulus polymers with a microstructured surface can provide a pull force greater than 50 Newtons, where the normal force is 50 Newtons, according to the present COSA protocol.

It should be appreciated that a distinction between a single phase coefficient of friction and two phase coefficient of friction can be identified. The standard physics description of the coefficient of friction, described by the equation above, includes only one solid phase in contact with a second solid phase. However, novel and unexpected shear forces and peel forces are revealed when a third phase is included, such that the first and second solid phases are in contact with a phase of liquid or gas (third phase). The notion of coefficient of friction is not easily applied to the following configurations: solids connected through a gaseous or liquid phase, liquids connected through a solid or gaseous phase, and gases connected through a solid or liquid phase. These two-phase structures are the subject of the present invention.

Referring to Tables 4 and 5, structures of form given by FIG. 2 are tested in a two-phase (wet) embodiment. Comparing Tables 2 and 3 to Tables 4 and 5, for the one phase (dry) microstructured surfaces and the two-phase wet microstructured surfaces, the COSA shows the perimeter of the surface of contact scales linearly with the shear force for dry microstructured surfaces where for the two-phase microstructured surfaces the COSA does not scale with contact area or perimeter length. Furthermore, the COSA is greater than a magnitude higher for wet microstructured devices of the present invention and dry microstructured devices.

Tables 4 and 5 illustrate a new phenomenon not understood heretofore and cannot technically be described as a coefficient of friction. The effect, discovered here, has very little to do with the surface area or perimeter length, but instead geometry of the microstructure may play a dominant role in the microstructured device's resistance to translation when the target surface is coated with a liquid.

This effect is associated with a metastable interfacial state called a Wenzel-Cassie interface. This interface in contact with the present microstructures is unexpected, and may reveal a wet interface between a microstructured surface and a target surface that displays non-classical physical shear force phenomena.

TABLE 4
Wet (N = 10)

| Microstructure | COSA (shear force/normal force) |
|---|---|
| PLA sheet no microstructure | 0.35 +/− 0.11 |
| 100% area of contact | |
| PLA sheet 100% | 31.4 +/− 4.5 |
| microstructure <25% | |
| area of contact | |
| PLA 90% | 23.5 +/− 3.4 |
| PLA 80% | 12.9 +/− 3.3 |
| PLA 70% | 10.5 +/− 3.3 |
| PLA 60% | 9.0 +/− 2.2 |
| PLA 50% | 7.0 +/− 2.2 |
| PLA 40% | 3.0 +/− 2.1 |
| PLA 30% | 1.1 +− 1.0 |
| PLA 20% | 0.57 +/− 0.15 |
| PLA 10% | 0.29 +/− 0.15 |

TABLE 5
COSA, Wet

| Contact Area | Perimeter area | COSA |
|---|---|---|
| 1 | 1 | 30.4 +/− 0.21 |
| 1 | 0.8 | 29.1 +/− 0.36 |
| 1 | 0.5 | 16.8 +/− 0.27 |
| 1 | 0.3 | 11.5 +− 0.16 |

In practice, a true solid on solid interface is never realized. For example, objects in everyday use carry condensation, such as liquids, oils, and the like. These layers can be microscopic, but they may have macro effects. For example, microstructure patterns on gripping pad surfaces, rotating gripping surfaces, fingertips of gloves, brake pads, clutch plates, working surfaces of pliers, graspers, retractors, swabs, robotic tools, and laparoscopic surgical devices all involve a second phase, typically a liquid or flowable medium.

Gripping surfaces can be of the form tapes or films or layers formed on working surfaces, for example, handles for tools and coating surfaces such as labels, closures, surfaces for utensils, and everyday objects that typically contact liquids. In many cases, the microstructured surfaces may be designed for a variety of second phase interfaces, such as water, surfactants, oils, or combinations of these. The grip strength of the microstructure surfaces of the present invention may be enhanced by liquids that can be structured by an interface. For example, in some embodiments, water will form a hydrogen bonded microstructured liquid in response to van der Waals forces. In some embodiments, oils will form enclosing structures. And in some embodiments, amphiphilic structures, surfactants, can form biological structures, such as cell walls. All of these structures can be induced synthetically on a microstructured surface with the appropriate range of contact angles. These contact angles may be designed to vary over the hierarchical structure of the microstructure. The microstructure surfaces may be organized such that having alternating high and low surface energies, wherein the high surface energy may generate a flowing domain and a low surface energy may generate a locking or constrained domain. The combination of a flowing and a locking domain is referred to as the Wenzel-Cassie interface.

The reason microstructured surfaces may have a higher dry COSA compared to non-microstructured surfaces is because the shear force may be dominated by the increase in perimeter length due to the microstructure in contact with the target surface.

For hierarchical microstructured structures, the underlying layers of microstructure may not be in contact (for rigid target surfaces) with the target surface and may not contribute to the dry COSA. Thus, the perimeter length may not necessarily be increased, and can even be decreased. Nevertheless, hierarchical microstructured surfaces in contact with a wet interface may exhibit shear forces that may be insensitive to the perimeter length, and as disclosed here the shear force may be greater than any of the dry shear forces for various perimeter lengths.

For example, referring to Tables 6 and 7, where the second level of microstructure is flattened to the first layer, and the contact area is kept constant by stretching and compression of the microstructure, it is shown that the two-level hierarchical structure providing regions of hydrophilic surface regions juxtaposed hydrophobic surface regions has a higher COSA than one-level surfaces in contact with a wet target surface. Note all values are normalized to dry, two-level force of 1. In these embodiments, there is essentially no difference between one-level and two-level surfaces when dry, although the soft surface is able to deform into the microstructure, which augments the second level relative to a one-level device. Tables 6 and 7 should not be compared in absolute terms, since the normalization was different in the two cases. Within Tables 6 and 7, one-level wet has a lower COSA than one-level dry (the classical result).

TABLE 6
COSA, Dry, Steel Grips

| Contact Area | Perimeter area | microstructure geometry | COSA |
|---|---|---|---|
| Dry | | | |
| 1 | 1 | two-level | 1.00 +/− 0.15 |
| 1 | 1 | one-level | 0.92 +/− 0.69 |
| Wet | | | |
| 1 | 1 | two-level | 25.4 +/− 0.57 |
| 1 | 1 | one-level | 0.47 +− 0.18 |

TABLE 7
COSA, Dry, Silicon Rubber Grips

| Contact Area | Perimeter area | microstructure geometry | COSA |
|---|---|---|---|
| Dry | | | |
| 1 | 1 | two-level | 1.00 +/− 0.31 |
| 1 | 1 | one-level | 0.69 +/− 0.52 |
| Wet | | | |
| 1 | 1 | two-level | 18.9 +/− 0.39 |
| 1 | 1 | one-level | 0.30 +− 0.27 |

In certain embodiments disclosed therein, it has been found that despite the differences due to the modulus of the target surface, as illustrated in Tables 6 and 7, the COSA for two-level hierarchical surfaces including a liquid interface for a target surface are consistently higher by more than one order of magnitude than two-level hierarchical surfaces in a dry interface. For dry interfaces, the difference between one-level and two-level microstructured surfaces is small, but for wet/liquid interfaces there is a large difference.

It is expected that in some embodiments, the water and gas interface, or in the case of an in vivo interface a water and lipid interface, the two components of the liquid interface may segregate into philic and phobic domains. That is, droplets of water may adhere to the hydrophilic, high surface energy regions of the microstructure, and the spheres of gas or lipid may adhere to the hydrophobic, low surface energy regions of the microstructure. This type of philic-phobic segregated interface, known as a Wenzel-Cassie interface, may be understood to be an energetically lower state than an interface that is a mixture of gas and liquid, or water and lipid. When a microstructured surface capable of generating the Wenzel-Cassie wetting states is translated relative to a target surface that resides between the two surfaces, then the energy required for translation may be greater than for an interface where the philic-phobic components are not segregated. Consequently, a microstructure may order a liquid interface with an arbitrarily stiff target surface which exhibits a COSA at least one order of magnitude above the COSA obtained with a gas only interface.

It has been observed that a water only interface may not exhibit such enhanced gripping effects disclosed herein. For microstructure surfaces having different surface energy, i.e., hierarchical or distributed, water alone may organize via hydrogen bonding into high energy domains and low energy domains. Such segregation is not visually apparent and has been referred to in the literature as structured water.

The enhanced gripping effect of embodiments disclosed herein relies upon an organizational or conformational change in the interfacial composition between the microstructure and the target surface, and such organizational effects may result in a COSA at least one order of magnitude greater than effects attributed to surface of contact area and/or perimeter length.

Of interest, one may consider the standard understanding regarding the static or kinetic COF between two surfaces when there is a liquid interface, as shown in Table 8.

TABLE 8
Reduction of COF due to fluidic interface.

| | | Static Coefficient of Friction | |
|---|---|---|---|
| | | Dry | Lubricated |
| Material | Against Material | Contact | Contact |
| Aluminum | Aluminum | 1.10-1.35 | 0.30 |
| Aluminum-Bronze | Steel | .45 | — |
| Alloy | | | |
| Aluminum | Steel | .61 | — |
| Brake (Composite) | Cast Iron | .40 | .21 (wet) |
| Brass | Steel | .50 | .19 |
| Brass | Cast Iron | .28 | |
| Brick | Wood | .60 | — |
| Bronze | Cast Iron | .21 | — |
| Bonze Sintered | Steel | — | .12 |
| Bronze | Steel | | .16 |
| Cadmium | Cadmium | .50 | .05 |
| Cadmium | Chromium | .40 | .35 |
| | (Chrome) | | |
| Cadmium | Steel, Mild | .45 | |
| Carbon | Carbon | .15 | .12-.14 |
| Carbon | Steel | .14 | .12-.14 |
| Cast Iron | Cast Iron | 1.1 | .08 |
| Cast Iron | Copper | 1.05 | — |
| Cast Iron | Oak Wood | .485 | .08 |
| Cast Iron | Steel | .40 | .21 |
| Cast Iron | Zinc | .85 | — |
| Chromium | Chromium | .41 | .34 |
| (Chrome) | (Chrome) | | |
| Cobalt (70° C.) | Cobalt | .3 | — |
| Concrete | Rubber | 1.0 | .30 (wet) |
| Concrete | Wood | .62 | — |
| Copper | Cast Iron | 1.05 | — |
| Copper | Copper | 1.00 | .08 |
| Copper | Glass | .94 | — |
| Copper | Steel | .53 | — |
| Copper-Lead Alloy | Steel | .22 | — |
| Cork | Iron | .35 | — |
| Diamond | Diamond | .10 | .05-.10 |
| Diamond | Metal | .10-.15 | .10 |
| Glass | Glass | .90-1.0 | .10-.60 |
| Glass | Metal | .50-.70 | .20-.30 |
| Glass | Nickel | .78 | .57 |
| Graphite | Graphite | .10 | .10 |
| Graphite | Steel | .1 | .1 |
| Horseshoe | Concrete | .67 | — |
| Horseshoe | Rubber | .28 | — |
| Ice | Wood | .05 | — |
| Ice | Steel | .04 | — |
| Iron | Iron | 1.0 | .15-.20 |
| Lead | Cast Iron | .41 | — |

Embodiments disclosed herein having a microstructured surface may not fall within the ranges of CoFs disclosed in Table 8. In particular, embodiments disclosed herein comprising microstructured surfaces may generate COSA which are greater than 2.0 COSA.

### Example 1. A Microstructure that Generates a Wenzel-Cassie Interface

Surface energy is generally understood in the art as the contact angle of a droplet of water when placed on a surface. Since the water drop interacts with one or more layers of microstructure, the established interfacial layer between a microstructured surface, a fluidic layer, and a target surface can be complicated. This example is intended to enable one in the art to generate an enhanced target COSA between a microstructured surface and a range of target surfaces.

One may appreciate one or more of the parameters regarding a microstructured surface, which include, but are not limited to: 1) the pitch or distance between microstructure centers, 2) the area of the microstructures, and 3) the height of the microstructures.

The latter parameter may not be considered relevant to one not practiced in the present art. A microstructured surface with multiple stacked layers of microstructure presents a 3-dimensional surface to the environment. Such a surface may organize the environmental constituents as well as the target surface according to the distribution of differing surface energy surfaces.

For example, referring to FIG. 3, a microstructured surface 300 comprises first microstructure level 302, second microstructure level 304 and third microstructure level 306. There may be lateral microstructures 308. Such a structure has a surface energy distribution both in the plane 310 and the normal 312. For example, first region 314 has a different surface energy than second region 316. Accordingly, interfacial fluid 318 comprises philic components 320 and phobic components 322, which are associated respectively with high surface energy surfaces, such as the first region 314, and low surface energy surfaces, such as the second region 316. Furthermore, the regions of philic components 320 and phobic components 322 are three dimensional in nature. Note that philic components 320 being of different surface energy than phobic components 322 will not pass through each other easily, and may therefore require substantial energy to do so, which is interpreted as a high static coefficient of friction.

The embodiment illustrated in FIG. 3 may be understood to generate a Wenzel-Cassie interface when a liquid is present at the interface of the target surface and microstructured surface.

### Example 2. Relation Between Contact Angle and Pitch for Microstructured Surfaces

In order to design hierarchical microstructured surfaces with regions of different surface energy (contact angle) it may be beneficial to understand the relation between the pitches of the microstructures and contact angle.

Referring now to FIG. 2, an embodiment is illustrated wherein the surface comprises a polymer, such as polylactic acid for example, and the microstructure features have a pitch which may be altered by heated equilibrium stretching. The water contact angle may be measured as a function of pitch. Theoretically, the shape of a liquid-vapor interface may be determined by the Young-Dupré equation, with the contact angle playing the role of a boundary condition via the Young equation.

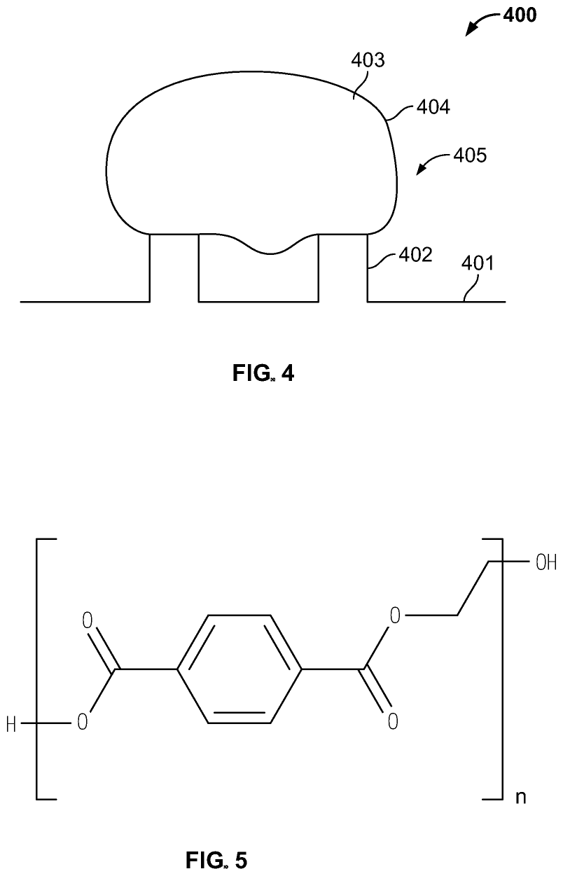

The theoretical description of contact arises from the consideration of a thermodynamic equilibrium between the three phases: the liquid phase, the solid phase, and the gas or vapor phase (which could be a mixture of ambient atmosphere and an equilibrium concentration of the liquid-vapor). Referring now to FIG. 4, a microstructure surface 400 may include a surface 401 having disposed thereon microstructure features 402. A liquid 404 may contact the microstructure features 402 such that the liquid “sits” on the top portions of the microstructure features in some embodiments. The liquid phase 403 and gas phase 405 may have an effect on the thermodynamic equilibrium depending on the microstructure pattern and the interaction of all three phases.

The “gaseous” phase could be replaced by another immiscible liquid phase such as a lipid phase. Referring to FIG. 4, the solid-vapor interfacial energy is denoted by γ_{SG}, the solid-liquid interfacial energy by γ_{SL}, and the liquid-vapor interfacial energy (i.e. the surface tension) by γ_{LG}, then the equilibrium contact angle θ_{C} is determined from these quantities by the Young equation:

$${γ}_{SG} - {γ}_{SL} - {γ}_{LG} cos {θ}_{C} = 0$$

Regarding dimensional nomenclature, for practical reasons, pitch p will be measured and reported as the distance between first level pillars not the distance from pillar center to pillar center (the usual definition). The standard pitch dimension P is related to the measured pitch dimension p by: P=p+D where D is the first level pillar diameter.

Embodiments disclosed herein with varied pitch may be produced by spray mounting the embodiment of FIG. 1 on a chilled copper plate and then slowly heating to about 40° C., allowing it to equilibrate, and then rapidly chilling to 0° C., where the pattern can easily be released from the spray mount. This may produce a uniform pitch change in both x and y directions. Increasing pitch dimensions can be achieved by multiple applications of this technique. Test regions were microscopically selected such that the pattern under the test drop was uniformly dilated.

Using water, the contact angle (degrees) X was measured relative to pitch Y. A line fit to the data, for embodiments illustrated in FIG. 2, yielded:

$$Y = 83.4 exp [ - 1.3 \times 1 {0}^{- 2} X ]$$

### Example 3. Effect of Contact Angle on Living Cells

Embodiments of microstructured surfaces disclosed herein may be useful as implants in the body because they may provide resistance to migration when wet. Living cells are good probes of surface energy as a function of pitch or distance between surface microstructures. In addition to immobilizing implants in situ, microstructured surfaces may be used to promote healing.

One can measure the adherence of cells to a microstructured substrate by measuring their resonance frequency with respect to the microstructured surface. Epithelial cells were cultured on the electrospun microstructured surfaces and flat surfaces of the same material.

Resonant vibrations of individual epithelial cells supported on a surface were measured using a simple optical detection technique. A stream of saline was used to apply an impulse to the epithelial cells and their time dependent resonant oscillations were monitored by passing a laser beam through the cell and measuring the variations of the intensity of the scattered light using a fiber optic terminated spectrometer. The resulting time dependent intensity changes were Fourier transformed using software supplied with the spectrometer to obtain information about the vibrational frequencies of the droplets. The resonant frequencies of cells were obtained on surfaces with water contact angles ranging from 55 to 72.5 degrees. A contact angle dependence of the resonant frequency of the cells exposed to a steady stream was measured.

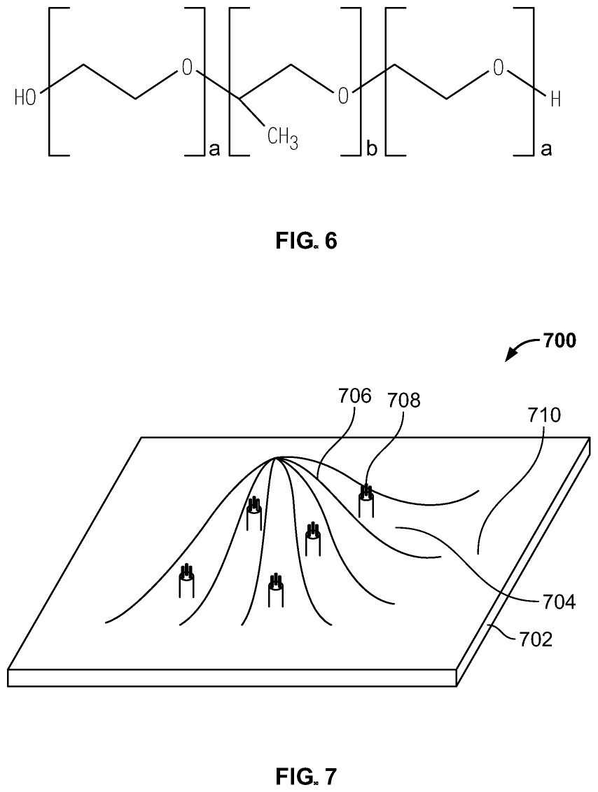

Embodiments having microstructured surfaces were constructed using PET with the chemical structure given in FIG. 5 and having a contact angle of about 72.5 degrees. The PET was blended with a polyurethane poloxamer of the form in FIG. 6. The polyurethane poloxamer has a contact angle of about 55 degrees.

PET was slowly added to the polyurethane poloxamer in solution until a contact angle of 55.3, 60.7, 65.5, 70.1 and 72.5 were obtained.

Plotting the cellular frequency Y against contact angle X obtains:

$$Y = 61.5 exp [ - 2.5 \times {10}^{- 2} X ]$$

Combining with the Pitch Vs Contact Angle equation given above, then

$$Pitch ( microns ) = 83.4 exp [ - 0.012 {θ}_{c , pitch} ]$$

$$and$$

$$Freq . ( Hz ) = 61.5 exp [ - 0.025 \text{?} ]$$

$$\text{?} \text{indicates text missing or illegible when filed}$$

where the contact angle for pitch is assumed close to the contact angle for the material which then gives a relationship between the expected cellular adhesion (Freq.) as a function of the microstructure pitch.

Pitch(microns)=2.19Freq.(Hz)

gives a relation between the cellular tone and pitch for a given substrate material. The tone is to be understood as a resonance frequency, somewhat independent of the flow that induces such tone.

For epithelial cells on flat surfaces: Lower material surface energy (lower contact angle) may correlate with lower resonance frequency. On microstructure surfaces: closer spacing (smaller pitch) may correlate with lower surface energy (more hydrophobic, higher contact angle). In conclusion, closer spacing of pillars (smaller pitch) may produce lower resonance frequency (more loosely bound) between cells and a given microstructured surface type.

This result may be understood as surprising given that it is the opposite of what is expected. Such results may be valid only for pitch near the dimensions of the cell, i.e., pitch in the range 5-100 microns. The result might be explained by the fact that the surface energy of the cell wall may be dominant on a low energy surface, and therefore may be less responsive to the underlying microstructure. This may suggest that the free cell resonant frequency may be below 1 Hz. Thus, a high energy microstructured surface may couple the cell to its environment more strongly.

### Example 4. A Microstructured Device for Gripping Low COSA Target Surfaces Comprising Multiple Microstructures

Referring to FIG. 7, a microstructured device 700 may comprise a surface 702, first microstructure 704, second microstructure 706, and third microstructure 708. First microstructure 704 may comprise a sinusoidal surface. Second microstructure 706 may comprise a series of radial ridges disposed hierarchically on the first microstructure surface 704 as well as also disposed non-hierarchically 710 on the surface 702. Third microstructure 708 may comprise pillars that are disposed on a portion of the first microstructure 704.

### Example 5. A Microstructured Device for Gripping Low COSA Target Surfaces Comprising Multiple Hierarchically Arranged Microstructures

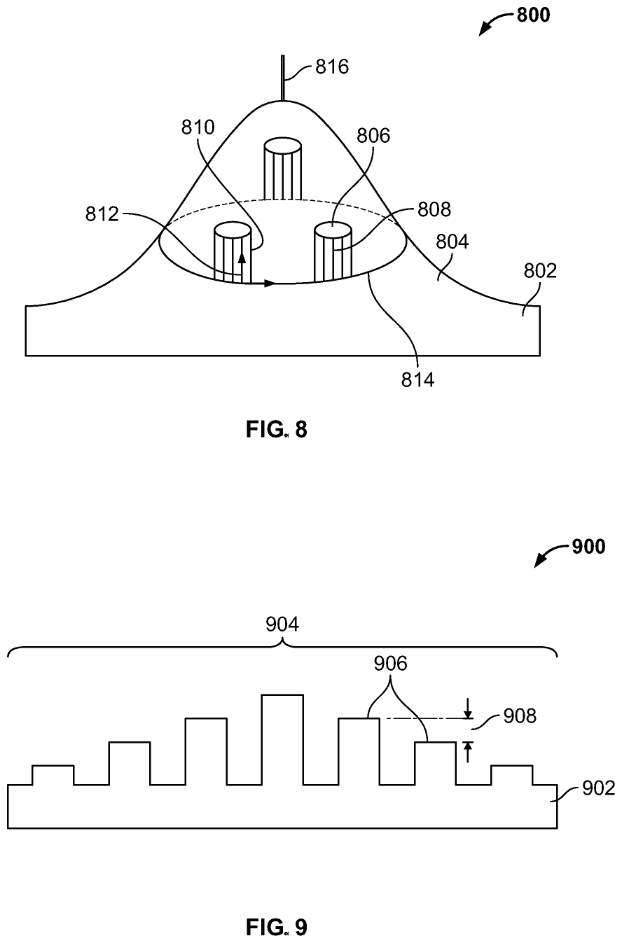

Referring to FIG. 8, a microstructured device 800 may comprise a surface 802, first microstructure 804, second microstructure 806, and third microstructure 808. First microstructure 804 may comprise a sinusoidal surface. Second microstructure 806 may comprise pillars that are disposed hierarchically on the first microstructure 804. Third microstructure may comprise a series of ridges 810 disposed hierarchically on second microstructure 806 such that the direction of the ridges 812 on each second microstructure 806 may be tangent to a circle 814 parallel to substrate 802 with its center also the center of a sinusoid peak 816.

### Example 6. A Microstructured Device for Gripping Low COSA Target Surfaces Comprising Two Microstructure Sets Wherein One Microstructured Set Comprises the Other Microstructured Set

Referring to FIG. 9, a microstructured device 900 comprises substrate 902, first microstructure 904, and second microstructure 906. First microstructure 902 may comprise second microstructures 906 having a graduated height 908. Second microstructures 906 may comprise the pillars of first microstructure 904 embodied as a tetrahedron.

### Example 7. A Microstructured Device for Gripping Low COSA Target Surfaces Comprising a Frictional Aspect

It will be appreciated that Examples 4-6 may be directed toward surfaces which obtain a higher COSA at a wet interface.

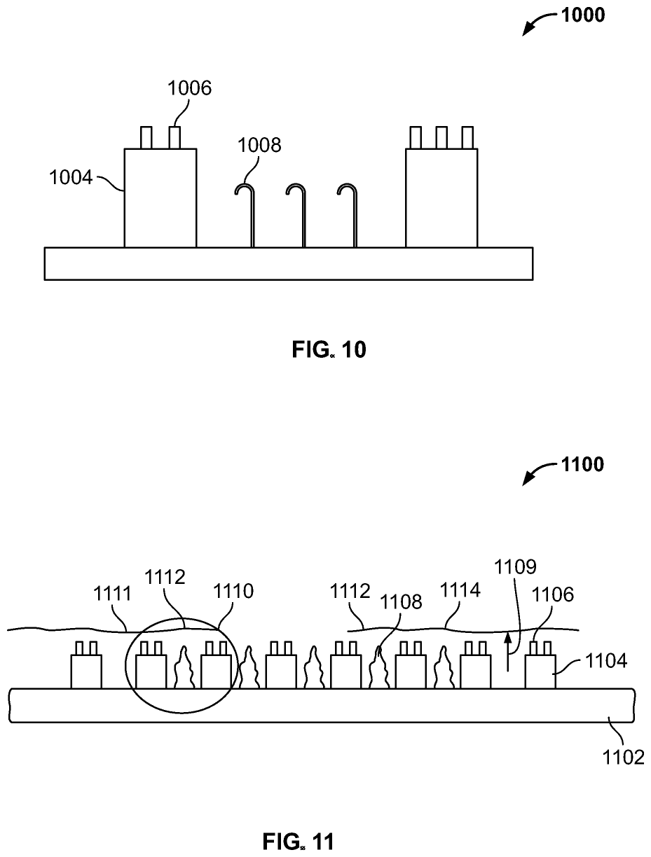

Referring now to FIG. 10, a microstructured device 1000 may comprise a surface, first microstructure 1004, second microstructure 1006, and third microstructure 1008. First microstructure 1004 may comprise rectangular pillars. Second microstructure 1006 may comprise smaller rectangular pillars that are disposed of on the first microstructure 1004. Third microstructure 1008 may comprise a series of hooked fibers disposed between a plurality of said first microstructure 1004 such that said hooked fibers of third microstructure 1008 readily attach to irregular surfaces when dry and lay down when wet.

### Example 8. A Microstructured Device for Gripping Low COSA Target Surfaces Comprising a Liquid Sealing Aspect

Referring now to FIG. 11, a microstructure device 1100 may comprise a surface 1102, a first microstructure 1104, a second microstructure 1106, and a third microstructure 1108, the third microstructure may be capable of contouring to fill a defect. First microstructure 1104 and second microstructure 1106 form a hierarchically arranged compound pillar that may be capable of generating a suctional force when configured in regular arrays and placed in contact with a wet surface. Third microstructure 1108 may be disposed in sealing area 1110 that may be capable of swelling when wet. The volume of swell 1112 can cause third microstructure 1108 to exceed the height of second microstructure 1106 and/or the height of the first microstructure 1104 and second microstructure 1106. Thus, the third microstructure 1108 may form a sealing area 1110 between microstructured device 1100 and target surface 1114.

In some cases, the Young's modulus of the material may be higher, equal to, or lower than the target surface, depending on the interface composition and the degree of delicacy of the target surface.

In some embodiments, the Young's modulus may include any Young's modulus capable of a solid form. In particular, the gripping surface may not be of high Young's modulus, or even a modulus higher than the target surface. Thus, embodiments having a gripping surface disclosed herein may not have a Young's modulus higher than the material to be gripped, although grip force in general does increase with Young's modulus, wherein the gripping material is sufficiently compliant to the target surface. When the Young's modulus of the microstructured surface is less compliant, then shear force may be compromised by insufficient area of contact.

In fact, in many instances of embodiments disclosed herein, the gripping surface may have a Young's modulus less than the target contacting surface, in at least one aspect of the pattern of the present microstructured surface.

Thus, although there have been described particular embodiments of the present invention of a new and useful TWO-PHASE ADHESIVE NONSTANDARD COSA DEVICES it is not intended that such references be construed as limitations upon the scope of this invention except as set forth in the following claims.

What is claimed is:

1. A microstructured device for gripping low coefficient of friction material comprising: a substrate, the substrate further comprising a gripping surface, the gripping surface comprising a first microstructure and a second microstructure, wherein the first microstructure and second microstructure comprise protuberances or voids, the protuberances or the voids being arranged to generate a Wenzel-Cassie state when in contact with a target surface.

2. The microstructured device of claim 1, wherein the gripping surface further comprises a third microstructure.

3. The microstructured device of claim 1, wherein the protuberances or the voids are in the shape of a cylinder, cone, sphere, or polytope.

4. The microstructured device of claim 1, wherein the first microstructure is a two-dimensional sinusoid microstructure.

5. The microstructured device of claim 1, wherein the first microstructure and the second microstructure are configured with a percentage contact area of the target surface of 50% or less.

6. The microstructured device of claim 1, wherein the gripping surface comprises a coefficient of static adhesion in excess of 3.

7. The microstructured device of claim 1, wherein the second microstructure is hierarchically disposed of on the first microstructure.

8. The microstructured device of claim 1, wherein the first microstructure and second microstructure are disposed with a pitch of 1 to 1000%, a height of 1 to 1000 microns, and a diameter of 1 to 1000 microns.

9. The microstructured device of claim 8, wherein the first microstructure is disposed of with a pitch of 45 to 185%, a height of 50 to 250 microns, and a diameter of 35 to 125 microns.

10. The microstructured device of claim 1, wherein at least one of the first microstructure and second microstructure gradually tapers to the target surface.

11. The microstructured device of claim 10, wherein at least one of the first microstructure and second microstructure gradually taper as described by a trigonometric function.

12. The microstructure device of claim 1, wherein the surface area of the gripping surface is 10 times or more than the surface area of the substrate.

13. The microstructure device of claim 2, wherein the third microstructure comprises a series of hooked fibers.

14. The microstructure device of claim 13, wherein the series of hooked fibers are disposed of between the first microstructure and the second microstructure.

15. The microstructure device of claim 2, wherein the third microstructure is capable of swelling when wet.

16. The microstructure device of claim 15, wherein the third microstructure exceeds the height of the second microstructure and/or the height of the first microstructure and second microstructure when wet.

17. The microstructure device of claim 2, wherein the third microstructure comprises a series of parallel ridges disposed hierarchically on the second microstructure.

18. The microstructure device of claim 1, wherein the first microstructure comprises pillars of graduated heights.

19. A microstructured device for gripping low coefficient of friction material comprising: a substrate, the substrate further comprising a gripping surface, the gripping surface comprising a first microstructure, a second microstructure, and a third microstructure, wherein the first microstructure, the second microstructure, and the third microstructure comprise protuberances or voids, the protuberances or the voids being arranged to generate a Wenzel-Cassie state.
